(19) United States
(12) Patent Application Publication
(10) Pub. No.: US 2021/0265020 A1
(43) Pub. Date: Aug. 26, 2021

(54) HIGH-PRECISION ENERGY RANKING METHOD USED FOR CRYSTAL STRUCTURE PREDICTION OF ORGANIC MOLECULES

(71) Applicant: SHENZHEN JINGTAI TECHNOLOGY CO., LTD., Guangdong (CN)

(72) Inventors: Yingdi JIN, Guangdong (CN); Qun ZENG, Guangdong (CN); Peiyu ZHANG, Guangdong (CN); Jian MA, Guangdong (CN); Lipeng LAI, Guangdong (CN); Shuhao WEN, Guangdong (CN)

(73) Assignee: SHENZHEN JINGTAI TECHNOLOGY CO., LTD., Guangdong (CN)

(21) Appl. No.: 16/472,206

(22) PCT Filed: May 4, 2018
(86) PCT No.: PCT/CN2018/085726

§ 371 Date: Jun. 21, 2019

(51) Int. Cl. G16C 10/00; G16C 20/30; G16C 20/50

(52) U.S. Cl. CPC G16C 10/00; G16C 20/50; G16C 20/30

(57) ABSTRACT

A high-precision energy ranking method used for the crystal structure prediction of organic molecules, including: determining a quantum mechanical radius of a center cell; carrying out an energy calculation in the center cell through a density fragment interaction algorithm; calculating an interaction energy of the molecules outside the center cell acting on the molecules in the center cell within the radius R under quantum mechanical precision; calculating an interaction energy of peripheral extension cells beyond the radius R acting on the molecules in the center cell under molecular mechanical precision; and calculating a total crystal energy.

### TECHNICAL FIELD

The invention belongs to the technical field of crystal structures of organic molecules, and particularly relates to a high-precision energy ranking method used for the crystal structure prediction of organic molecules.

### DESCRIPTION OF RELATED ART

The characteristic of compounds to form different crystal structures is called polymorphism, and the crystal form of the compounds has a great influence on their key physical and chemical properties such as density, morphology, solubility and dissolution rate. As for drugs, the crystal form can strongly affect the drug bioavailability, which in turn affects the treatment performance of the drugs. Experimental polymorphic drug screening has become an essential link to be conducted during standard drug research and development. In experiments, experimenters set key crystallization parameters manually or by means of robots, but correct crystallization conditions are difficult to obtain in a short time by experimentations. One feasible solution to this problem is to carry out crystal structure prediction (CSP) on drug molecules by computer simulation so as to search out various potential stable crystal forms, and then tested on a few targeted potential crystal forms.

In the past ten years, significant progress has been made in inorganic CSP and organic CSP. In spite of their similarities, inorganic CSP and organic CSP face distinctly different challenges. As for inorganic CSP, what people are concerned with are bonding and electron properties, while in the organic CSP, people are concerned more about structural changes as well as phase changes. The CSP of organic molecules is involved in drug research, and two major challenges are currently encountered in this field: one challenge is the sampling integrity of crystal space, and the other challenge is the accuracy of energy ranking of crystal structures. A great improvement has been fulfilled for the first challenge, while accurate ranking of the crystal structures still remains as a bottleneck that restrains mass application of CSP to the pharmaceutical industry.

Accurate calculation of small energy differences between low-energy crystal structures depends on high-precision quantum mechanical calculation, and the time complexity of high-precision quantum mechanical calculation reaches O(N³)—O(N⁴) according to the increase of the number N of system electrons, and when the number of molecules in an asymmetric unit increased and with an increase in the operands of space groups, a high-precision energy calculation of a large quantity of crystal structures generated in the CSP process becomes a bottleneck for CSP.

### BRIEF SUMMARY OF THE INVENTION

In view of the above technical problems, one objective of the invention is to improve the energy ranking efficiency and accuracy of crystal structures by providing a method for rapidly and accurately calculating the energy of a large quantity of crystal structures generated during the CSP of organic molecules. To fulfill the above objective, a density block interaction algorithm is used for energy calculation of an isolated macromolecule cluster system and QM/MM used for a periodic system are combined to design an efficient and accurate energy calculation method suitable for a periodic organic molecule crystal system. A framework designed by the calculation method is applicable to a variety of different precision first principle calculation methods of quantum mechanics, functional, and basis sets.

The technical solution adopted by the invention is as follows:

A high-precision energy ranking method used for crystal structure prediction of organic molecules includes the following steps:

(1) determining a quantum mechanical radius of a center cell

A Van der Waals radius sum of atoms in molecules in the center cell and atoms in molecules in a peripheral extension cell is calculated, a circle of molecules closest to the center cell is searched out with the addition of the radius sum and 1.5 Å as a cut-off, and a maximum distance from a geometric center of the center cell to the circle of molecules is taken as the quantum mechanical radius R;

(2) carrying out an energy calculation in the center cell through a density fragment interaction method

The energy of each of the molecules is calculated under precision of quantum mechanics, wherein the energy includes an electrostatic potential which is generated by nucleus potentials and electron density distribution of the rest of the molecules, the electrostatic potential and the electron density distribution of the molecules are obtained by iterative convergence, and the energy obtained after iterative convergence is as follows:

$${E}_{iso} [ ρ ] = {\sum }_{i} {E}_{i}^{0} [ {ρ}_{i} ] + \frac{1}{2} {\sum }_{i , j} ( {E}_{NN}^{i , j} - \int \int \frac{{ρ}_{i} {ρ}_{j}}{\vert \vec{r} - \vec{{r}^{′}} \vert} d \vec{r} d \vec{{r}^{′}} ) + Δ {E}_{xc} + Δ {T}_{s}$$

Eᵢₛₒ[ρ] refers to an energy in the center cell, wherein ρ represents the electron density, the energy in the center cell consists of three parts: the first part is the sum of the energy of each of the isolated molecules, wherein Eᵢ⁰[ρᵢ] refers to an energy of a molecule i, and ρᵢ refers to an electron density of the molecule i; the second part is the electrostatic interaction in between the molecules and includes the electrostatic interaction energy E_{NN}^{i,j} of the nucleuses of the molecule i and the molecule j, and the electrostatic interaction energy

$$\int \int \frac{{ρ}_{i} {ρ}_{j}}{\vert \vec{r} - \vec{{r}^{′}} \vert} d \vec{r} d \vec{{r}^{′}}$$

of the electron densities of the molecule i and the molecule j; the third part includes a nonlinear superposition error ΔEₓₑ of an exchange-correlation function between the molecules and a nonlinear superposition error ΔTₛ of a kinetic function between the molecules respectively; the parameters in each of the following formulae have the same symbols as those in this formula;

(3) calculating an interaction energy of the molecules outside the center cell acting on the molecules in the center cell within the radius R under the precision of quantum mechanics, by continuing the iterative process in Step 2, but only the interaction energy of the molecules outside the center cell acting on the molecules in the center cell is calculated:

$${E}_{inter \_ ij \_ QM} = {E}_{NN}^{i , j} - \int \int \frac{{ρ}_{i} {ρ}_{j}}{\vert \vec{r} - \vec{{r}^{′}} \vert} d \vec{r} d \vec{{r}^{′}}$$

The interaction energy E_{inter_ij_QM} of the molecules outside the center cell acting on the molecules in the center cell includes electrostatic interaction between nucleuses and interaction between electrons;

(4) calculating an interaction energy of periodic extension cells beyond the radius R acting on the molecules in the center cell, and under precision of the classical molecular mechanics. The energy is obtained by integrating an overall electron density in the center cell with a long-range electrostatic potential generated at the center cell by the molecules in the periodic extension cells, wherein a calculation formula is as follows:

E_{inter_ij_MM}=∫ρᵢV_{j_period}^{esp}d

[symbol]

Wherein, V_{jperiod}^{esp} refers to the electrostatic potential, beyond the radius R, generated at the center cell by the molecule j and all periodic mirror molecules of the molecule j, and ρᵢ refers to the electron density of the molecule i in the center cell;

(5) calculating a total crystal energy, the total crystal energy includes the energy of the center cell and the energy between the center cell and the periodic extension cell:

E=Eᵢₛₒ+E_{periodic}

Wherein, the energy calculated in Step (2) is taken as the energy of the center cell, and the energy of the periodic extension cells acting on the center cell is the sum of the energy calculated in Step (3) and the energy calculated in Step (4):

$${E}_{periodic} = \frac{1}{2} ( {\sum }_{{i = 1}_{( {R}_{ij} < R ) ⋂ ( j ∉ center \_ cell )}}^{n , m} {E}_{inter \_ ij \_ QM} + {\sum }_{{i = 1}_{( {R}_{ij} > R )}}^{n , m} {E}_{inter \_ ij \_ MM} )$$

Wherein, E_{inter_ij_QM} refers to the energy calculated in Step (3), the summation condition (Rᵢⱼ<R)∩(j∉center_cell) refers to a distance between the molecule i and the molecule j is smaller than the radius R determined in Step (1) and the molecule j is not within the center cell, and E_{inter_ij_MM} refers to the energy calculated in Step (4).

The high-precision energy ranking method used for crystal structure prediction of organic molecules has the following technical effects:

(1) The energy calculation efficiency of the molecule crystals of the drug is improved, and the computational complexity of the crystal structures on the precision of quantum mechanical level is reduced to O(N) from O(N³)—O(N⁴);

(2) A correct crystal searching direction and accurate energy feedback are ensured, and during the process of CSP, CSP will be guided to find out the true low-energy potential crystal form in the correct direction.

### BRIEF DESCRIPTION OF THE DRAWINGS

FIG. 1 is a schematic diagram for a quantum mechanical radius determined in one embodiment;

FIG. 2 is a flow diagram of a density fragment interaction method in the embodiment.

### DETAILED DESCRIPTION OF THE INVENTION

The specific technical solution of the invention is explained below in combination with embodiments.

A high-precision energy ranking method used for the crystal structure prediction of organic molecules includes the following steps:

(1) A quantum mechanical radius of a center cell is determined

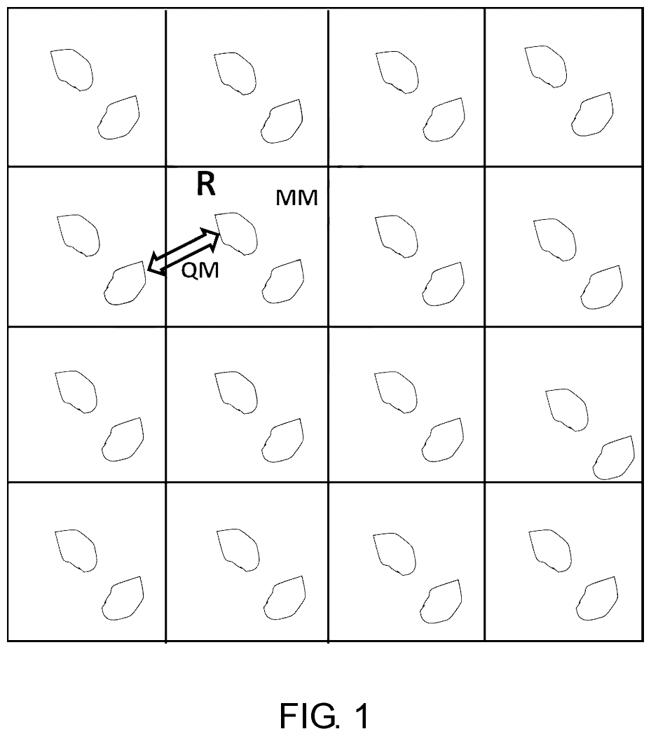

A Van der Waals radius sum of atoms in molecules in the center cell and atoms in molecules in a peripheral extension cell is calculated, a circle of molecules closest to the center cell is searched out with the addition of the radius sum and 1.5 Å as a cut-off, and a maximum distance from a geometric center of the center cell to the circle of molecules is taken as the quantum mechanical radius R; as shown in FIG. 1;

(2) Energy calculation is carried out in the center cell through a density fragment interaction method

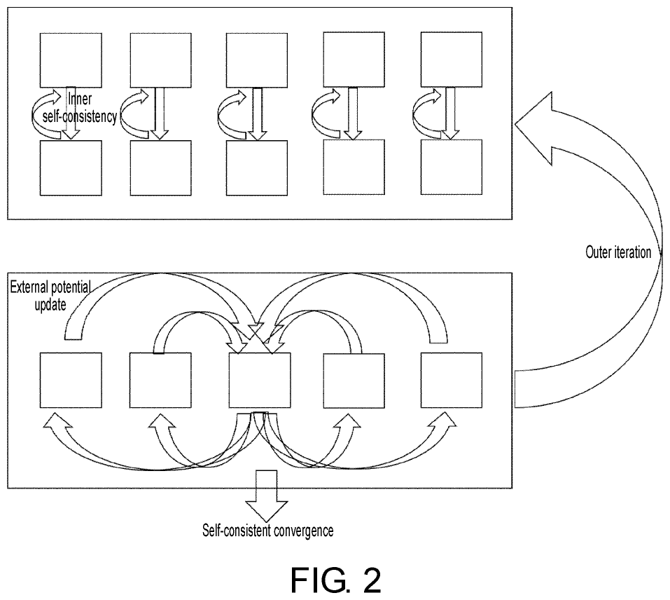

The energy of each of the molecules is calculated under precision of quantum mechanics, wherein the energy includes an electrostatic potential which is generated by nucleus potentials and electron density distribution of the rest of the molecules, and the electrostatic potential and the electron density distribution of the molecules are obtained by iterative convergence; as shown in FIG. 2; the energy obtained after iterative convergence is as follows:

$${E}_{iso} [ ρ ] = {\sum }_{i} {E}_{i}^{0} [ {ρ}_{i} ] + \frac{1}{2} {\sum }_{i , j} ( {E}_{NN}^{i , j} - \int \int \frac{{ρ}_{i} {ρ}_{j}}{\vert \vec{r} - \vec{{r}^{′}} \vert} d \vec{r} d \vec{{r}^{′}} ) + Δ {E}_{xc} + Δ {T}_{s}$$

Eᵢₛₒ[ρ] refers to an energy in the center cell, wherein ρ represents the electron density, the energy in the center cell consists of three parts: the first part is the sum of the energy of each of the isolated molecules, wherein Eᵢ⁰[ρᵢ] refers to an energy of a molecule i, and A refers to an electron density of the molecule i; the second part is the electrostatic interaction in between the molecules and includes the electrostatic interaction energy E_{NN}^{i,j} of nucleuses of the molecule i and the molecule j and the electrostatic interaction energy

$$\int \int \frac{{ρ}_{i} {ρ}_{j}}{\vert \vec{r} - \vec{{r}^{′}} \vert} d \vec{r} d \vec{{r}^{′}}$$

of the electron densities of the molecule i and the molecule j; the third part includes a nonlinear superposition error ΔEₓₑ of an exchange-correlation function between the molecules and a nonlinear superposition error ΔTₛ of a kinetic function between the molecules respectively; the parameters in each of the following formulae have the same symbols as those in this formula;

(3) calculating an interaction energy of the molecules outside the center cell acting on the molecules in the center cell within the radius R under the precision of quantum mechanics precision, as shown in FIG. 1, and by continuing the iterative process in Step 2, but only the interaction energy of the molecules outside the center cell acting on the molecule in the center cell is calculated:

$${E}_{inter \_ ij \_ QM} = {E}_{NN}^{i , j} - \int \int \frac{{ρ}_{i} {ρ}_{j}}{\vert \vec{r} - \vec{{r}^{′}} \vert} d \vec{r} d \vec{{r}^{′}}$$

Wherein the interaction energy E_{inter_ij_QM} of the molecules outside the center cell acting on the molecules in the center cell includes electrostatic interaction between the nucleuses and interaction between electrons;

(4) The interaction energy of the periodic extension cells beyond the radius R acting on the molecules in the center cell is calculated under precision of the classical molecular mechanics. The energy is obtained by integrating an overall electron density in the center cell with a long-range electrostatic potential generated at the center cell by the molecules in the periodic extension cells, wherein a calculation formula is as follows:

E_{inter_ij_MM}=∫ρᵢV_{j_period}^{esp}d

[symbol]

Wherein, V_{jperiod}^{esp} refers to the electrostatic potential, beyond the radius R, generated at the center cell by the molecule j and all periodic mirror molecules of the molecule j, and ρᵢ refers to the electron density of the molecule i in the center cell;

(5) A total crystal energy is calculated, the total crystal energy includes the energy of the center cell and the energy between the center cell and the periodic extension cells:

E=Eᵢₛₒ+E_{periodic}

Wherein, the energy calculated in Step (2) is taken as the energy of the center cell, and the energy of the periodic extension cells acting on the center cell is the sum of the energy calculated in Step (3) and the energy calculated in Step (4):

$${E}_{periodic} = \frac{1}{2} ( {\sum }_{{i = 1}_{( {R}_{ij} < R ) ⋂ ( j ∉ center \_ cell )}}^{n , m} {E}_{inter \_ ij \_ QM} + {\sum }_{{i = 1}_{( {R}_{ij} > R )}}^{n , m} {E}_{inter \_ ij \_ MM} )$$

Wherein, E_{inter_ij_QM} refers to the energy calculated in Step (3), the summation condition (Rᵢⱼ<R)∩(j∉center_cell) refers to a distance between the molecule i and the molecule j is smaller than the radius R determined in Step (1) and the molecule j is not within the center cell, and E_{inter_ij_MM} refers to the energy calculated in Step (4).

1. A high-precision energy ranking method used for crystal structure prediction of organic molecules, comprising the following steps:
Step (1) determining a quantum mechanical radius of a center cell,
wherein, a Van der Waals radius sum of atoms in molecules in the center cell and atoms in molecules in peripheral extension cells is calculated, a circle of molecules closest to the center cell is searched out with the addition of the radius sum and 1.5 Å as a cut-off, and a maximum distance from a geometric center of the center cell to the circle of molecules is taken as the quantum mechanical radius R;
Step (2) carrying out an energy calculation in the center cell through a density fragment interaction method,
wherein, the energy of each of the molecules in the center cells is calculated under precision of quantum mechanics, wherein the energy calculation of the current molecule includes an electrostatic potential which is generated by nucleus potentials and electron density distribution of the other molecules in the center cell, the electrostatic potential and the electron density distribution of the molecules are obtained by an iterative convergence method, and the energy obtained after the iterative convergence method is shown as the following Formula 1:
$${E}_{iso} [ ρ ] = {\sum }_{i} {E}_{i}^{0} [ {ρ}_{i} ] + \frac{1}{2} {\sum }_{i , j} ( {E}_{NN}^{i , j} - \int \int \frac{{ρ}_{i} {ρ}_{j}}{\vert \vec{r} - \vec{{r}^{′}} \vert} d \vec{r} d \vec{{r}^{′}} ) + Δ {E}_{xc} + Δ {T}_{s} , \qquad ( Formula 1 )$$
wherein, Eᵢₛₒ[ρ] refers to an energy in the center cell, ρ represents an electron density, Eᵢ⁰[ρᵢ] refers to an energy of a molecule i, and ρᵢ refers to an electron density of the molecule I, wherein the energy in the center cell consists of three parts: a first part is a sum of energy of each of the molecules in the center cell; a second part is an electrostatic interaction in between the molecules and comprising an electrostatic interaction energy E_{NN}^{i,j} of nucleuses of the molecule i and a molecule j, and an electrostatic interaction energy
$$\int \int \frac{{ρ}_{i} {ρ}_{j}}{\vert \vec{r} - \vec{{r}^{′}} \vert} d \vec{r} d \vec{{r}^{′}}$$
of the electron densities of the molecule i and the molecule j; and a third part includes a nonlinear superposition error ΔE_{xc} of an exchange-correlation function between the molecules, and a nonlinear superposition error ΔTₛ of a kinetic function between the molecules respectively, wherein parameters in each off the following formulae have the same symbols as those shown in the above Formula 1;
Step (3) calculating an interaction energy of the molecules outside the center cell acting on the molecules in the center cell within the radius R under precision of quantum mechanics,
wherein, by continuing the iterative convergence method as shown in the Step (2), wherein only the interaction energy of the molecules outside the center cell acting on the molecules in the center cell is calculated as shown in the following Formula 2:
$${E}_{inter \_ ij \_ QM} = {E}_{NN}^{i , j} - \int \int \frac{{ρ}_{i} {ρ}_{j}}{\vert \vec{r} - \vec{{r}^{′}} \vert} d \vec{r} d \vec{{r}^{′}} , \qquad ( Formula 2 )$$
wherein, the interaction energy E_{inter_ij_QM} of the molecules outside the center cell acting on the molecules in the center cell includes the electrostatic interaction between the nucleuses and the interaction between electrons;
Step (4) calculating an interaction energy of the peripheral extension cells beyond the radius R acting on the molecules in the center cell, and under precision of classical molecular mechanics,
wherein, the interaction energy is obtained by integrating an overall electron density in the center cell with a long-range electrostatic potential generated at the center cell by the molecules in the peripheral extension cells,
wherein a calculation formula is shown as the following Formula 3:
E_{inter_ij_MM}=∫ρᵢV_{j_period}^{esp}d [symbol] (Formula 3),
wherein, V_{jperiod}^{esp} refers to the electrostatic potential, beyond the radius R, generated at the center cell by the molecule j and all periodic mirror molecules of the molecule j, and ρᵢ refers to the electron density of the molecule i in the center cell; and
Step (5) calculating a total crystal energy,
wherein, the total crystal energy includes the energy Eᵢₛₒ in the center cell and an energy E_{periodic} between the center cell and the peripheral extension cells as shown in the following Formula 4:
E=Eᵢₛₒ+E_{periodic} (Formula 4),
wherein, the energy calculated in the Step (2) is taken as the energy of the center cell, and the energy of the peripheral extension cells acting on the center cell is the sum of the energy calculated in the Step (3) and the energy calculated in the Step (4) as shown in the following Formula 5:
$${E}_{periodic} = \frac{1}{2} ( {\sum }_{{i = 1}_{( {R}_{ij} < R ) ⋂ ( j ∉ center \_ cell )}}^{n , m} {E}_{inter \_ ij \_ QM} + {\sum }_{{i = 1}_{( {R}_{ij} > R )}}^{n , m} {E}_{inter \_ ij \_ MM} ) , \qquad ( Formula 5 )$$
wherein, E_{inter_ij_QM} refers to the energy calculated in the Step (3), a summation condition (Rᵢⱼ<R)∩(j∉center_cell) refers to a distance between the molecule i and the molecule j is smaller than the radius R determined in the Step (1) and the molecule j is not within the center cell, and E_{inter_ij_MM} refers to the energy calculated in the Step (4).
